SEQUENCE LISTING (6 sequences)

SEQ ID NO: 1 - protein, 40 aa, Pholiota squarrosa
  MISC_FEATURE (10)..(10): X stands for any amino acid, preferably Cys.
  MISC_FEATURE (17)..(17): X stands for any amino acid, preferably Cys.
  MISC_FEATURE (20)..(20): X stands for Tyr/Ser.
  MISC_FEATURE (23)..(23): X stands for Phe/Tyr.
  MISC_FEATURE (27)..(27): X stands for Arg/Lys/Asn.
  MISC_FEATURE (33)..(33): X stands for Asp/Gly/Ser.
  MISC_FEATURE (35)..(35): X stands for Asn/Ala.
  MISC_FEATURE (39)..(39): X stands for Thr/Gln.
APVPVTKLVXDGDTYKXTAXLDXGDGXWVAQWXTXVFHXG

SEQ ID NO: 2 - protein, 39 aa, Stropharia rugosoannulata Farlow in Murr.
  MISC_FEATURE (4)..(4): X stands for Pro/Gly.
  MISC_FEATURE (7)..(7): X stands for Glu/Lys.
  MISC_FEATURE (9)..(9): X stands for Val/Asp.
  MISC_FEATURE (10)..(10): X stands for any amino acid, preferably Cys.
  MISC_FEATURE (13)..(13): X stands for Asn/Asp/Glu.
  MISC_FEATURE (17)..(17): X stands for any amino acid, preferably Cys.
  MISC_FEATURE (20)..(20): X stands for His/Ser.
  MISC_FEATURE (27)..(27): X stands for Lys/His.
  MISC_FEATURE (29)..(29): X stands for Val/Ile.
  MISC_FEATURE (33)..(33): X stands for Gly/Asn/Ser.
  MISC_FEATURE (34)..(34): X stands for Ala/Thr.
  MISC_FEATURE (39)..(39): X stands for Arg/Thr.
APVXVTXLXXDGXSYKXTAXLDYGDGXWXAQWXXNVFHX

SEQ ID NO: 3 - protein, 40 aa, Lepista sordida (Schum. : Fr.) Sing.
  MISC_FEATURE (1)..(1): X stands for Ala/Gln.
  MISC_FEATURE (4)..(4): X stands for Pro/Lys.
  MISC_FEATURE (7)..(7): X stands for Ala/Ser.
  MISC_FEATURE (8)..(8): X stands for Met/Ile/Val.
  MISC_FEATURE (9)..(9): X stands for Tyr/Thr.
  MISC_FEATURE (10)..(10): X stands for any amino acid, preferably Cys.
  MISC_FEATURE (13)..(13): X stands for Asp/Asn.
  MISC_FEATURE (16)..(16): X stands for Lys/Glu.
  MISC_FEATURE (17)..(17): X stands for any amino acid, preferably Cys.
  MISC_FEATURE (20)..(20): X stands for Ala/Asn.
  MISC_FEATURE (22)..(22): X stands for Val/Asp/Asn.
  MISC_FEATURE (25)..(25): X stands for Asp/Asn.
  MISC_FEATURE (27)..(27): X stands for Arg/His/Asn.
  MISC_FEATURE (31)..(31): X stands for Gln/Arg.
  MISC_FEATURE (34)..(34): X stands for Thr/Val.
XPVXVKXXXXDGXTYXXTAXLXYGXGXWVAXWSXAVFHQS

SEQ ID NO: 4 - protein, 39 aa, Naematoloma sublateritium (Fr.) Karst/Hypholoma sublateritium(Fr.)Quel
  MISC_FEATURE (10)..(10): X stands for any amino acid, preferably Cys.
  MISC_FEATURE (13)..(13): X stands for Asp/Thr.
  MISC_FEATURE (14)..(14): X stands for Ser/Ala.
  MISC_FEATURE (16)..(16): X stands for Gln/Lys.
  MISC_FEATURE (17)..(17): X stands for any amino acid, preferably Cys.
APVPVTKLVXDGXXFXXTANLDFGDGNWVAQWSTNVFHN

SEQ ID NO: 5 - protein, 40 aa, Naematoloma sublateritium (Fr.) Karst/Hypholoma sublateritium(Fr.)Quel
  MISC_FEATURE (10)..(10): X stands for any amino acid, preferably Cys.
  MISC_FEATURE (14)..(14): X stands for Asp/Thr.
  MISC_FEATURE (15)..(15): X stands for Ser/Ala.
  MISC_FEATURE (17)..(17): X stands for Gln/Lys.
  MISC_FEATURE (18)..(18): X stands for any amino acid, preferably Cys.
APVPVTKLVXDDGXXFXXTANLDFGDGNWVAQWSTNVFHN

SEQ ID NO: 6 - protein, 39 aa, Artificial sequence
  Modification of SEQ ID(1) in which Ala at position 1, Tyr at position 20 and Thr at position 39 are substituted with Lys, and Gly at position 40 is deleted.
KPVPVTKLVCDGDTYKCTAKLDFGDGRWVAQWDTNVFHK

(19) United States
(12) Patent Application Publication
(10) Pub. No.: US 2020/0264182 A1
(43) Pub. Date: Aug. 20, 2020

(54) METHOD FOR DETECTING PROSTATE CANCER

(71) Applicants: J-OIL MILLS, INC., Tokyo (JP); OSAKA UNIVERSITY, Osaka (JP)

(72) Inventors: Kazutoshi FUJITA, Osaka (JP); Norio NONOMURA, Osaka (JP); Eiji MIYOSHI, Osaka (JP); Yuka KOBAYASHI, Tokyo (JP)

(21) Appl. No.: 16/644,545

(22) PCT Filed: Sep. 21, 2018
(86) PCT No.: PCT/JP2018/035155

§ 371 Date: Mar. 5, 2020

(30) Foreign Application Priority Data
Sep. 29, 2017 (JP) 2017-191505

(51) Int. Cl. G01N 33/574; G01N 33/68

(52) U.S. Cl. CPC G01N 33/57434; G01N 33/68

(57) ABSTRACT

[Problem] To provide a method for detecting prostate cancer, the method having a higher degree of certainty than the PSA blood test and providing measurement values that correlate with risk classification.

[Solution] This method for detecting prostate cancer is characterized in that: the method includes reacting fucose α1→6 specific lectin and fucosylated PSA contained in a sample constituted of serum collected from a subject, and detecting the reacted lectin; and the pH in at least one step of the group consisting of the reaction step for reacting the fucosylated PSA and the lectin and processing steps thereafter is adjusted to above 8.5 and less than 11.0.

### TECHNICAL FIELD

The present invention relates to a method for detecting prostate cancer and more specifically to the detection method using measurement of a serum fucosylated PSA.

### BACKGROUND ART

Prostate is a reproductive organ that is present just below a bladder of a male so as to surround his urethra. Prostate cancer has increased in recent years. Blood PSA screening is known as a method for screening prostate cancer. “PSA” means a prostate specific antigen. PSA is classified into isolated PSA (also referred to as “F-PSA” or “free PSA”) and complex PSA bound to α1-antichymotrypsin (ACT) (also referred to as “PSA-ACT”). A human blood PSA level is the sum of free PSA and complex PSA. Although a blood PSA level of a healthy person increases with age, its level is normally less than 4 ng/mL. If a person exhibits an abnormally high level of 4 ng/mL or higher, the person is suspected of prostate cancer and judged as test positive.

A person judged as test positive in PSA screening is subjected to prostate biopsy for definitive diagnosis. In this biopsy, a clinician samples tissues from a site suspected of prostate cancer using a prostate needle and subjects the tissues and cells to a histopathological test to classify the prostate cancer risk (malignancy and severity).

This risk classification is comprehensively determined by combining three factors: blood PSA level, Gleason score and staging (TNM classification). The Gleason score (hereinafter also referred to as GS) is an indicator for determining cancer malignancy in a tissue. Histological forms of cancers in collected specimens are classified into G1 to G5 patterns on the basis of situations of tissues and infiltration (Prostate Cancer Handling Regulation, 4th edition). Evaluation by G3 to G5 is as described below.

### Gleason Pattern

G3: consisting of independent gland duct having a distinct lumen, and infiltrating between existing non-neoplastic ducts.
G4: exhibiting fused gland duct, cribriform gland duct, hypemephromatoid, indistinct gland duct formation.
G5: exhibiting solid growth, trabecular conformation, arcuate growth, comedonecrosis.

Patterns of the most frequent lesions (dominant lesions) and the second most frequent lesions (accompanying lesions) are respectively judged, and the sum of their values is defined as the Gleason score (GS). The higher the Gleason score is, the higher the malignancy of the cancer is. Prostate cancers at GS 7 or higher are progressive and metastatic and are therefore called high-risk prostate cancers requiring early treatments such as surgery.

The TNM classification is an international standard for evaluating prostate cancer from the viewpoints of tumor size (T), metastasis to lymph node (N), and distant metastasis (M). Furthermore a stage (disease stage) is determined from the TNM classification.

The blood PSA level also increases due to aging, prostatic inflammation and prostatomegaly. Even if a patient with an abnormally high blood PSA level is biopsied, a detection rate of the prostate cancer cell is only about 30%. In addition, in view of the blood PSA levels and the GS of the prostate patient group shown in Table 6, patients having high blood PSA levels do not necessarily have a high prostate cancer malignancy.

Under such circumstances, it is desired that a method for detecting prostate cancer is developed with a detection accuracy of prostate cancer higher than that of blood PSA screening is desired. Furthermore, it is desired that the detection method correlates to the risk classification such as the Gleason score. If the risk can be classified and a high-risk prostate cancer can be predicted from measurement values, a frequency of biopsies can be reduced.

There have been some known reports that prostate cancer is detected on the basis of a cancerous change of a sugar chain binding to a PSA. For example, a PSA analysis method described in Patent Document 1 is characterized in that a lectin having an affinity for a fucose α1-2 galactose residue is brought into contact with a PSA-containing sample to determine an amount of the PSA having an affinity for the lectin. Prostate cancer and prostatomegaly are distinguished on the basis of a knowledge that an α1-2 fucosylated PSA increases in a specimen sampled from blood of a prostate cancer patient.

Non Patent Document 1 describes that a normal PSA includes few double-stranded asparagine-linked sugar chains (N-glycan) and mainly include hybrid-type and high mannose-type sugar chains, meanwhile a prostate cancer-derived PSA includes many branched N-glycans whose terminal binds to a sialic acid at α2-3.

Non-Patent Document 2 reports that when blood fucosylated PSAs of patients with prostate cancer or prostatomegaly having tested blood PSA levels of 4 to 10 ng/mL were measured by ELLA using a UEA-1 lectin having affinity for α1-2-fucose, fucosylated PSA levels in the prostate cancer patients were significantly higher than those in the patients with prostatomegaly.

Non-Patent Document 3 reports that, as a result of examining a blood fucosylated PSA level by an multiplex magnetic bead-based immunoassay using AAL, the blood fucosylated PSA level increased in association with increase of a GS. This document suggests that the blood fucosylated PSA level can be a surrogate biomarker for distinguishing between malignant prostate cancer and non-malignant prostate cancer.

Non-Patent Document 4 reports that, as a result of examining a urine fucosylated PSA level by a lectin antibody ELISA using AAL or PhoSL, the higher the GS of the prostate cancer patient was, the lower the urine fucosylated PSA level was. This document suggests that decrease in the urine fucosylated PSA level can be a marker for detecting a prostate cancer patient having a high GS.

### PRIOR ART DOCUMENTS

### Patent Documents

Patent Document 1: WO 2010/090264 A1 (Method for Analyzing PSA, and Method for Distinguishing between Prostate Cancer and Prostatomegaly Employing the Analysis Method)
Patent Document 2: JP Pat. No. 4514163 (fucose α1→6 specific lectin)
Patent Document 3: JP 2011-148736 A (peptide)

### Non-Patent Documents

Non-Patent Document 1: M. Tajiri et al., “Oligosaccharide profiles of the prostate specific antigen in free and complexed forms from the prostate cancer patient serum and in seminal plasma: a glycopeptide approach.”, Glycobiology vol. 18, No. 1, 2008, p 2-8
Non-Patent Document 2: Meriam V. Dwek et al., “A sensitive assay to measure biomarker glycosylation demonstrates increased fucosylation of prostate specific antigen (PSA) in patients with prostate cancer compared with benign prostatic hyperplasia”, Clinica Chimica Acta, 41, (2010), p 1935-1939
Non-Patent Document 3: Qing Kay Li et. al., “Serum Fucosylated Prostate-specific Antigen (PSA) Improves the Differentiation of Aggressive from Non-aggressive Prostate Cancers”, Theranostics.2015; 5(3): 267-276
Non-Patent Document 4: Kazutoshi Fujita et. al., “Decreased fucosylated PSA as a urinary marker for high Gleason score prostate cancer”, Oncotarget. 2016 Aug. 30; 7(35): 56643-56649
Non-Patent Document 5: Yuka Kobayashi et al., “A Novel Core Fucose-specific Lectin from the Mushroom Pholiota squarrosa”, J. Biol. Chem, 2012, 287, p 33973-33982

### SUMMARY OF INVENTION

### Problem to be Solved

An object of the present invention is to provide a method for detecting prostate cancer with a higher accuracy than that of the conventional PSA screening. Another object of the present invention is to provide a method for detecting prostate cancer in which measurement values correlate with a risk classification.

### Solution to Problem

As a result of intensive studies for solving the above problems, the present inventors have found that when a pH is adjusted to within a specific alkaline region in at least one step among a step for reacting a fucose α1→6 specific lectin with a serum fucosylated PSA and subsequent processing steps, a detection accuracy of prostate cancer is improved. The present inventors have found that the above problems can be solved on the basis of this finding, and this finding has led to completion of the present invention.

The present invention provides a method for detecting prostate cancer including reacting a fucosylated PSA contained in a specimen consisting of serum sampled from a subject with a fucose α1→6 specific lectin to detect the reacted lectin. This method is characterized in that a pH is adjusted to higher than 8.5 and lower than 11.0 in at least one step of the group consisting of a step for reacting the fucosylated PSA with the lectin and subsequent processing steps.

The methods disclosed in Non-Patent Documents 1 and 2 do not detect the α1→6 fucose sugar chain and are thus clearly different from the detection method according to the present invention. In Non-Patent Document 3, a serum fucosylated PSA level is measured using AAL. As described in the following Comparative Example 2, AAL has a high non-prostate cancer detection rate (false positive rate). On the other hand, in the present invention, the prostate cancer detection rate (positive rate) is high and the non-prostate cancer detection rate (false positive rate) is low so that it is superior to the method in Non-Patent Document 3. In Non-Patent Document 4, a urine fucosylated PSA level is measured using PhoSL. The method according to the present invention measures the serum fucosylated PSA and is thus different from the method in Non-Patent Document 4. In Non-Patent Document 4, a signal based on a complex of the fucosylated PSA and PhoSL decreases as the GS increases. Thus, subjects in Non-Patent Document 4 are persons suspected of prostate cancer because of a high blood PSA level previously measured. On the other hand, the method according to the present invention, in which subjects are not limited to persons suspected of prostate cancer, is used as an initial health screening.

The fucose α1→6 specific lectin is extracted from a basidiomycete belonging to, for example, Strophariaceae, Tricholomataceae, Amanitaceae or Polyporaceae.

The fucose α1→6 specific lectin is at least one of, for example, Pholiota squarrosa lectin, Pholiota terrestris lectin, Stropharia rugosoannulata lectin, Naematoloma sublateritium lectin, Lepista sordida lectin, and Amanita muscaria lectin.

For example, the fucose α1→6 specific lectin is a protein or peptide which contains:

(a) a protein or peptide consisting of an amino acid sequence represented by any of SEQ ID Nos. 1 to 5; or

(b) a protein or peptide consisting of an amino acid sequence represented by any of SEQ ID Nos. 1 to 5 in which one or more amino acids are deleted, inserted or substituted, and which is functionally equivalent to a protein having the amino acid sequence represented by any of SEQ ID Nos. 1 to 5.

The fucose α1→6 specific lectin may be labeled.

The fucosylated PSA may be detected by an assay using the fucose α1→6 specific lectin and an antibody.

The reaction between the fucosylated PSA and the fucose α1→6 specific lectin is preferably performed in the presence of albumin.

When a subject shows a signal (reaction value) resulting from the reaction between the fucosylated PSA and the fucose α1→6 specific lectin higher than a signal (reference value) obtained from a person with a Gleason score of 6 or lower, it is suggested that the subject has a high-risk prostate cancer. In this specification, the “high-risk prostate cancer” means a progressive prostate cancer with the Gleason score of 7 or higher.

The present invention also provides a diagnostic agent for detecting prostate cancer, containing: a fucose α1→6 specific lectin; and an alkaline reagent for adjusting a pH to higher than 8.5 and lower than 11.0 in at least one step selected from a group of steps including a step for reacting a fucosylated PSA contained in a specimen consisting of serum sampled from a subject with the fucose α1→6 specific lectin and subsequent processing steps.

Preferably, the diagnostic agent for detecting prostate cancer further contains an anti-PSA antibody.

### Effects of Invention

Since the blood PSA level based on the PSA screening also increases due to aging as well as non-prostate cancers such as prostatomegaly and prostatitis, the detection accuracy of prostate cancer based on the blood PSA level is low. On the other hand, in the detection method according to the present invention is characterized in that the step for reacting the serum fucosylated PSA with the fucose α1→6 specific lectin and the subsequent processing steps are executed in a specific alkaline region. Therefore, the detection method according to the present invention can detect prostate cancer with high accuracy, as demonstrated in the following Examples.

In the conventional PSA screening, it was difficult to distinguish between high-malignant prostate cancer and low-malignant prostate cancer. A person having a high blood PSA level and suspected of prostate cancer should have been evaluated in a risk classification such as Gleason score by biopsy. On the other hand, in the method according to the present invention, the higher the risk grade (malignancy) of the prostate cancer is, the higher the reaction value of the blood fucosylated PSA-fucose α1→6 specific lectin complex is. In other words, the reaction value and the risk grade (malignancy) correlate with each other.

The detection method of the present invention is expected to be non-invasive but be easy to extract the patients who should be essentially treated. The method of the present invention can also provide useful information about the presence or absence of prostate cancer, prior to prostate biopsy on a patient of test positive in the blood PSA test. For a patient having a blood PSA level in the gray zone, the degree of the detected level is an indicator for determining necessity of the biopsy. Also, for a patient having a blood PSA level remarkably higher than the standard value, the degree of the detected reaction value level is an indicator for distinction of the malignancy.

### BRIEF DESCRIPTION OF DRAWINGS

FIG. 1 is a structural drawing of an α1→6 fucose oligosaccharide and a non-α1→6 fucose oligosaccharide.

FIG. 2 is another structural drawing of the α1→6 fucose oligosaccharide and the non-α1→6 fucose oligosaccharide.

### DESCRIPTION OF EMBODIMENTS

Hereinafter, embodiments of the present invention will be explained in more detail. In the method for detecting prostate cancer according to the present invention (hereinafter, referred to as the method according to the present invention), a fucose α1→6 specific lectin is made to actuate on a fucosylated PSA contained in a specimen consisting a human serum under a specific condition to measure a signal (reaction value) based on a complex of the fucosylated PSA and the fucose α1→6 specific lectin.

A first candidate subject for the method according to the present invention is a human male who considers undergoing a PSA screening as a medical examination. The detection method according to the present invention detects prostate cancer with higher accuracy than that in the PSA screening, as demonstrated in the following Examples.

A second candidate subject for the method of the present invention is a person who exhibits an abnormally high blood PSA level of 4 ng/mL or more in a PSA screening. When a subject exhibits a blood PSA level of 4 ng/mL or more, he is suspected of prostate cancer and determined to be test-positive. Test-positive patients include patients who do not need treatment such as GS 6 patients and patients with advanced cancer such as GS 7 to 8 patients. In the method of the present invention, the reaction value of a complex of the fucosylated PSA and the fucose α1→6 specific lectin increases as the stage of prostate cancer progresses. Therefore, the method of the present invention can provide an indication for determining necessity of biopsy and information about cancer malignancy of a patient.

In Particular, a patient with a blood PSA level of 4-20 ng/mL is unlikely to have prostate cancer even if he is test-positive, or the patient is unlikely to require treatment because of being at GS 6 even if he suffers from prostate cancer. The method of the present invention can provide an indication of determining necessity of biopsy for such a patient.

The fucose α1→6 specific lectin can be defined by:

(1) a lower limit of a binding constant for the α1→6 fucose sugar chain; and

(2) an upper limit of a binding constant for sugar chains and glycolipid-type sugar chains excluding the α1→6 fucose.

More specifically, the fucose α1→6 specific lectin has all of the following properties [1]-[3].

[1] The fucose α1→6 specific lectin has affinity expressed by a binding constant of 1.0×10⁴ M⁻¹ or more (at 25° C.) for an α1→6 fucose sugar chain No. 405 having the following structural formula (1):

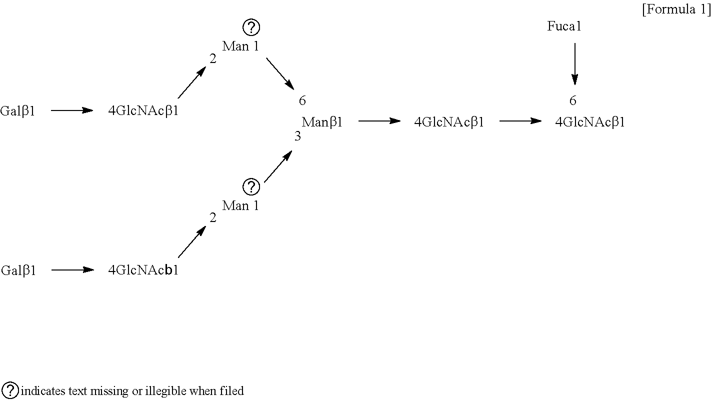

[wherein Gal, GlcNAc, Man and Fuc refer to galactose, N-acetylglucosamine, mannose and fucose respectively.]
[2] The fucose α1→6 specific lectin has a binding constant of 1.0×10³ M⁻¹ or less (at 25° C.) for a sugar chain No. 003 excluding the α1→6 fucose and having the following structural formula (2):

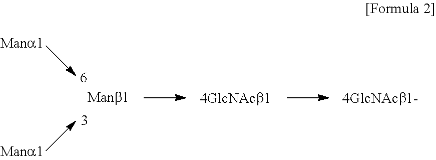

[wherein GlcNAc and Man refer to N-acetylglucosamine and mannose respectively.]
[3] The fucose α1→6 specific lectin has a binding constant of 1.0×10³ M⁻¹ or less (at 25° C.) for the glycolipid-type sugar chain No. 909 excluding the α1→6 fucose and having the following structural formula (3):

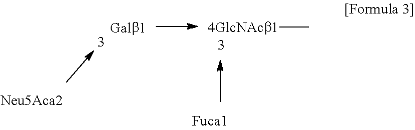

[wherein Gal, GlcNAc, Fuc and Neu5Ac refer to galactose, N-acetylglucosamine, fucose, and N-acetylneuraminic acid respectively.]

In this specification, the binding constant means a value measured e.g., by means of a frontal affinity chromatography (FAC method) at an analysis temperature of 25° C. Details of the FAC method are described in Patent Document 2 filed by some of the present applicants, for example.

The binding constant (at 25° C.) of the fucose α1→6 specific lectin for the α1→6 fucose sugar chain No. 405 is preferably 5.0×10⁴ M⁻¹ or more, more preferably 1.0×10⁵ M⁻¹ or more, still more preferably 2.0×10⁵ M⁻¹ or more.

The binding constant (at 25° C.) for the sugar chain No. 003 and glycolipid-type sugar chain No. 909 excluding the α1→6 fucose is generally 1.0×10³ M⁻¹ or less, preferably 1.0×10² M⁻¹ or less, particularly preferably 0.

Furthermore, the fucose α1→6 specific lectin may also have a high affinity for an α1→6 fucose sugar chain having a sialic acid at non-reduced terminal of the sugar chain No. 405. The term “high affinity” means that the binding constant (at 25° C.) is preferably 1.0×10⁴ M⁻¹ or more, more preferably 5.0×10⁴ M⁻¹ or more, and still more preferably 1.0×10⁵ M⁻¹ or more. On the other hand, some conventional lectins have a low affinity for the α1→6 fucose sugar chain having the sialic acid at the non-reduced terminal. Herein, the low affinity means that the binding constant (at 25° C.) is 1.0×10³ M⁻¹ or less.

The fucose α1→6 specific lectin further has an affinity expressed by a binding constant (at 25° C.) of preferably 1.0×10⁴ M⁻¹ or more, more preferably 5.0×10⁴ M⁻¹ or more, further preferably 1.0×10⁵ M⁻¹ or more for an N-linked single-, double-, triple- and/or quadruple-stranded sugar chain bound to the α1→6 fucose.

The molecular weight of the fucose α1→6 specific lectin based on SDS polyacrylamide electrophoresis is usually 4,000 to 40,000, preferably 4,000 to 20,000. Herein, the molecular weight based on SDS polyacrylamide electrophoresis is measured according to e.g. a method of Laemmi (Nature, vol. 227, page 680, 1976). The lectin may be generally formed by binding 2 to 10, preferably 2 to 6, more preferably 2 to 3 subunits to each other.

Fucose α1→6 specific lectins obtained from natural products will be outlined. The natural products are exemplified by mushrooms such as basidiomycetes and ascomycetes. Strophariaceae, Tricholomataceae, Polyporaceae and Amanitaceae belong to basidiomycetes. Examples of Strophariaceae include Pholiota squarrosa, Pholiota terrestris, Stropharia rugosoannulata, Naematoloma sublateritium, Pholiota aurivella, Pholiota adiposa and the like. Examples of Tricholomataceae include Lepista sordida and the like. Examples of Polyporaceae include Trichaptum elongatum, Microporus vemicipes and the like. Examples of Amanitaceae include Amanita muscaria and the like.

Methods of extracting and/or purifying the fucose α1→6 specific lectin from natural products are described in detail in Patent Document 2 filed by some of the present applicants and Non-Patent Document 5 submitted by the present applicants. Herein, Pholiota terrestris lectin (PTL) described in Patent Document 2 is replaced by Pholiota squarrosa lectin (PhoSL).

Among the above-mentioned basidiomycetes or ascomycetes, Strophariaceae, Tricholomataceae or Amanitaceae are preferred from the viewpoints of the specificity of the fucose α1→6 specific lectin for recognizing the α1→6 fucose sugar chain and the recovery efficiency of the lectin. Above all, Pholiota squarrosa lectin (PhoSL), Pholiota terrestris lectin (PTL), Stropharia rugosoannulata lectin (SRL), Naematoloma sublateritium lectin (NSL), Lepista sordida lectin (LSL) and Amanita muscaria lectin (AML) are particularly preferable. Amino acid sequences of the PhoSL, SRL, LSL and NSL are shown in Table 1.

The PhoSL shown in SEQ ID No. 1 is a lectin that can be extracted from Pholiota squarrosa. The Xaa at the 10th and 17th positions in SEQ ID No. 1 may be any amino acid residue, but is preferably Cys. The Xaa at the 20th, 23rd, 27th, 33rd, 35th and 39th positions are Tyr/Ser, Phe/Tyr, Arg/Lys/Asn, Asp/Gly/Ser, Asn/Ala and Thr/Gln, respectively.

The SRL shown in SEQ ID No. 2 is a lectin that can be extracted from Stropharia rugosoannulata. The Xaa at the 10th and 17th positions in SEQ ID No. 2 may be any amino acid residue, but is preferably Cys. The Xaa at the 4th, 7th, 9th, 13th, 20th, 27th, 29th, 33rd, 34th and 39th positions are Pro/Gly, Glu/Lys, Val/Asp, Asn/Asp/Glu, His/Ser, Lys/His, Val/Ile, Gly/Asn/Ser, Ala/Thr and Arg/Thr, respectively.

The LSL shown in SEQ ID No. 3 is a lectin that can be extracted from Lepista sordida. The Xaa at the 10th and 17th positions in SEQ ID No. 3 may be any amino acid residue, but is preferably Cys. The Xaa at the 1st, 4th, 7th, 8th, 9th, 13th, 16th, 20th, 22nd, 25th, 27th, 31st and 34th positions are Ala/Gln, Pro/Lys, Ala/Ser, Met/Ile/Val, Tyr/Thr, Asp/Asn, Lys/Glu, Ala/Asn, Val/Asp/Asn, Asp/Asn, Arg/His/Asn, Gln/Arg and Thr/Val, respectively.

The NSL shown in SEQ ID No. 4 is a lectin that can be extracted from Naematoloma sublateritium. The Xaa at the 10th and 17th positions in SEQ ID No. 4 may be any amino acid residue, but is preferably Cys. The Xaa at the 13th, 14th and 16th positions are Asp/Thr, Ser/Ala and Gln/Lys, respectively.

The NSL shown in SEQ ID No. 5 is also a lectin that can be extracted from Naematoloma sublateritium. The Xaa at the 10th and 18th positions in SEQ ID No. 5 may be any amino acid residue, but is preferably Cys. The Xaa at positions the 14th, 15th and 17th are Asp/Thr, Ser/Ala and Gln/Lys, respectively. Note that SEQ ID No. 5 can also be said to be a variant in which one Asn is inserted into the peptide of SEQ ID No. 4.

The fucose α1→6 specific lectin may be a protein or peptide which includes (a) a protein or peptide consisting of an amino acid sequence represented by any of SEQ ID Nos. 1 to 5; or (b) a protein or peptide consisting of an amino acid sequence represented by any of SEQ ID Nos. 1 to 5 in which one or more amino acids are deleted, inserted or substituted, and which is functionally equivalent to a protein having the amino acid sequence represented by any of SEQ ID Nos. 1 to 5.

The phrase “one or more amino acids are deleted, inserted or substituted” does not include amino acids added for other functions such as His tag, Flag tag, and GST tag, as well as spacers and the like for adding the amino acids. In addition, in a case of a protein or peptide in which a plurality of the sequences of (a) and (b) are linked, whether the phrase “one or more amino acids are deleted, inserted or substituted” is applied or not is determined with respect to a sequence corresponding to the (a) and (b), and spacers and the like for linking the amino acids are not related to the phrase “one or more amino acids are deleted, inserted or substituted”.

Herein, the “functionally equivalent” means that it has affinity expressed by a binding constant (at 25° C.) of 1.0×10⁴ M⁻¹ or more, preferably 5.0×10⁴ M⁻¹ or more, more preferably 1.0×10⁵ M⁻¹ or more, still more preferably 2.0×10⁵ M⁻¹ or more for the α1→6 fucose sugar chain No. 405. An example of a protein or peptide variant consisting of the amino acid sequence shown in SEQ ID No. 4 is a protein or peptide consisting of the amino acid sequence shown in SEQ ID No. 5.

The fucose α1→6 specific lectin may also be a peptide or protein obtained by not only extraction from the natural products but also chemical synthesis based on amino acid sequences of a naturally occurring lectin. Furthermore, the chemically synthesized peptide and protein may be a peptide in which one or several amino acids in amino acid sequences of a naturally occurring lectin are substituted with lysine and/or arginine and which has a carbohydrate-binding activity. A method for obtaining the peptides of the fucose α1→6 specific lectin is described in detail in Patent Document 3 filed by some of the present applicants. An amino acid sequence (SEQ ID No. 6) of the PhoSL peptide is shown in Table 1. The PhoSL peptide represented by SEQ ID No. 6 has an amino acid sequence in which the 1st Ala, the 20th Tyr, and the 39th Thr are substituted with Lys, and the 40th Gly is deleted in the specific example of PhoSL represented by SEQ ID No. 1 (APVPVTKLVC DGDTYKCTAY LDFGDGRWVA QWDTNVFHTG).

TABLE 1

| | | SEQ |
|---|---|---|
| | | ID |
| Lectin | Amino acid sequence | No. |
| PhoSL | Ala-Pro-Val-Pro-Val-Thr-Lys- | 1 |
| | Leu-Val-Xaa-Asp-Gly-Asp-Thr- | |
| | Tyr-Lys-Xaa-Thr-Ala-Xaa-Leu- | |
| | Asp-Xaa-Gly-Asp-Gly-Xaa-Trp- | |
| | Val-Ala-Gln-Trp-Xaa-Thr-Xaa- | |
| | Val-Phe-His-Xaa-Gly | |
| SRL | Ala-Pro-Val-Xaa-Val-Thr-Xaa- | 2 |
| | Leu-Xaa-Xaa-Asp-Gly-Xaa-Ser- | |
| | Tyr-Lys-Xaa-Thr-Ala-Xaa-Leu- | |
| | Asp-Tyr-Gly-Asp-Gly-Xaa-Trp- | |
| | Xaa-Ala-Gln-Trp-Xaa-Xaa-Asn- | |
| | Val-Phe-His-Xaa | |
| LSL | Xaa-Pro-Val-Xaa-Val-Lys-Xaa- | 3 |
| | Xaa-Xaa-Xaa-Asp-Gly-Xaa-Thr- | |
| | Tyr-Xaa-Xaa-Thr-Ala-Xaa-Leu- | |
| | Xaa-Tyr-Gly-Xaa-Gly-Xaa-Trp- | |
| | Val-Ala-Xaa-Trp-Ser-Xaa-Ala- | |
| | Val-Phe-His-Gln-Ser | |
| NSL | Ala-Pro-Val-Pro-Val-Thr-Lys- | 4 |
| | Leu-Val-Xaa-Asp-Gly-Xaa-Xaa- | |
| | Phe-Xaa-Xaa-Thr-Ala-Asn-Leu- | |
| | Asp-Phe-Gly-Asp-Gly-Asn-Trp- | |
| | Val-Ala-Gln-Trp-Ser-Thr-Asn- | |
| | Val-Phe-His-Asn | |
| NSL | Ala-Pro-Val-Pro-Val-Thr-Lys- | 5 |
| | Leu-Val-Xaa-Asp-Asp-Gly-Xaa- | |
| | Xaa-Phe-Xaa-Xaa-Thr-Ala-Asn- | |
| | Leu-Asp-Phe-Gly-Asp-Gly-Asn- | |
| | Trp-Val-Ala-Gln-Trp-Ser-Thr- | |
| | Asn-Val-Phe-His-Asn | |
| PhoSL | Lys-Pro-Val-Pro-Val-Thr-Lys- | 6 |
| Pep- | Leu-Val-Cys-Asp-Gly-Asp-Thr- | |
| tide | Tyr-Lys-Cys-Thr-Ala-Lys-Leu- | |
| | Asp-Phe-Gly-Asp-Gly-Arg-Trp- | |
| | Val-Ala-Gln-Trp-Asp-Thr-Asn- | |
| | Val-Phe-His-Lys | |

The fucose α1→6 specific lectin may be not only an extract from the natural product but also a recombinant artificially developed in a known host different from natural origins by using nucleic acids encoding an amino acid sequence of a naturally occurring lectin.

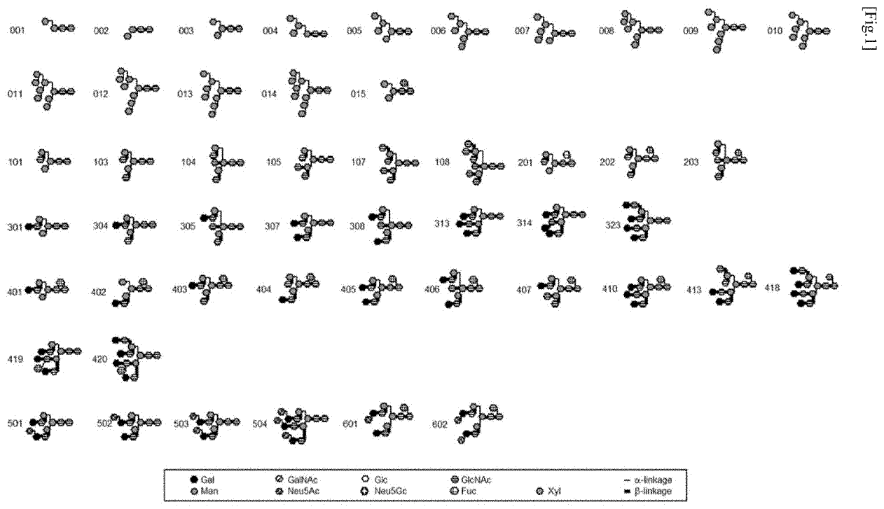

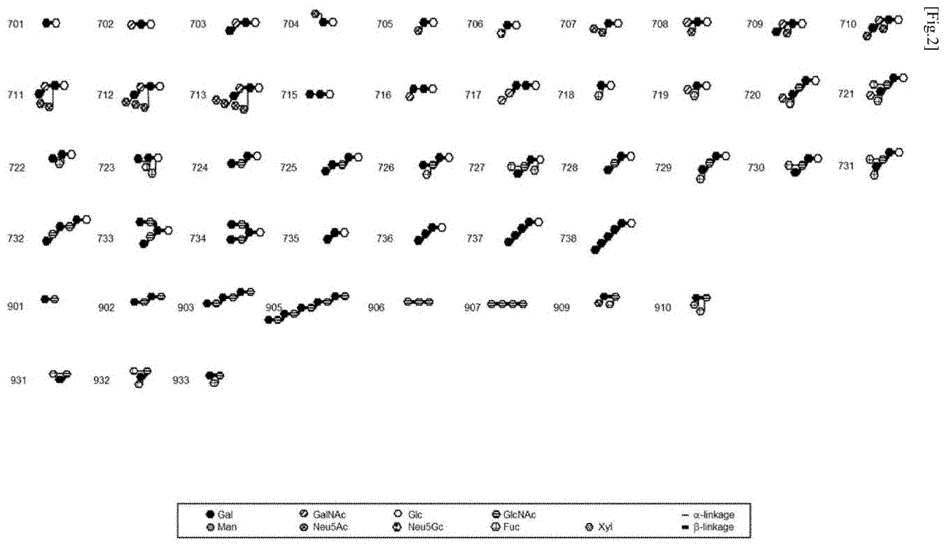

Binding constants (at 25° C.) of PhoSL, SRL, NSL and LSL belonging to the fucose α1→6 specific lectin for various sugar chains are shown in Tables 2 to 5. For comparison, binding constants (at 25° C.) of Aleuria aurantia lectin (AAL), Aspergillus oryzae lectin (AOL), Lens culinaris lectin (LCL), and Pisum sativum lectin (PSL) for various sugar chains (FIGS. 1 and 2) are also shown in Tables 2 to 5, these lectins having affinity for the fucose α1→6 but being not specific to the fucose α1→6.

TABLE 2
(1) Binding constant for sugar chains having α1→6 fucose (at 25° C.)

| Sugar | | | | | | | | |
|---|---|---|---|---|---|---|---|---|
| chain | PhoSL | SRL | NSL | LSL | AAL | AOL | LCL | PSL |
| No. | (M⁻¹) | (M⁻¹) | (M⁻¹) | (M⁻¹) | (M⁻¹) | (M⁻¹) | (M⁻¹) | (M⁻¹) |
| 015 | 5.0 × 10⁵ | 4.9 × 10⁴ | 3.6 × 10⁴ | 1.9 × 10⁵ | 9.1 × 10⁴ | 1.3 × 10⁵ | 5.6 × 10⁴ | 1.2 × 10⁵ |
| 201 | 4.6 × 10⁵ | 6.4 × 10⁴ | 3.8 × 10⁴ | 2.3 × 10⁵ | 5.1 × 10⁴ | 1.2 × 10⁵ | 5.0 × 10⁵ | 1.0 × 10⁵ |
| 202 | 4.0 × 10⁵ | 6.1 × 10⁴ | 3.9 × 10⁴ | 2.3 × 10⁵ | 5.4 × 10⁴ | 1.2 × 10⁵ | 1.1 × 10⁵ | 4.4 × 10⁴ |
| 203 | 3.3 × 10⁵ | 5.2 × 10⁴ | 3.0 × 10⁴ | 2.0 × 10⁵ | 6.6 × 10⁴ | 1.3 × 10⁵ | 7.3 × 10⁵ | 2.5 × 10⁴ |
| 401 | 3.5 × 10⁵ | 5.7 × 10⁴ | 5.1 × 10⁴ | 3.5 × 10⁵ | 5.5 × 10⁴ | 1.4 × 10⁵ | 4.2 × 10⁴ | 9.2 × 10⁴ |
| 402 | 2.0 × 10⁵ | 4.4 × 10⁴ | 3.6 × 10⁴ | 2.0 × 10⁵ | 1.1 × 10⁴ | 1.6 × 10⁵ | 5.9 × 10⁴ | 4.8 × 10⁴ |
| 403 | 3.4 × 10⁵ | 5.6 × 10⁴ | 5.2 × 10⁴ | 3.4 × 10⁵ | 5.2 × 10⁴ | 1.3 × 10⁵ | 9.9 × 10⁴ | 3.8 × 10⁴ |
| 404 | 3.9 × 10⁵ | 5.8 × 10⁴ | 4.3 × 10⁴ | 3.9 × 10⁵ | 6.5 × 10⁴ | 1.5 × 10⁵ | 5.8 × 10⁴ | 4.5 × 10⁴ |
| 405 | 3.2 × 10⁵ | 5.4 × 10⁴ | 5.0 × 10⁴ | 3.2 × 10⁵ | 5.6 × 10⁴ | 1.3 × 10⁵ | 4.7 × 10⁴ | 3.6 × 10⁴ |
| 406 | 2.2 × 10⁵ | 3.8 × 10⁴ | 2.3 × 10⁴ | 2.2 × 10⁵ | 4.7 × 10⁴ | 1.1 × 10⁵ | 1.8 × 10⁴ | 1.3 × 10⁴ |
| 407 | 2.8 × 10⁵ | 1.0 × 10⁴ | N.T | 2.8 × 10⁵ | 4.5 × 10⁴ | 1.1 × 10⁵ | 1.0 × 10³ | <1.0 × 10³ |
| 410 | 2.2 × 10⁵ | 3.9 × 10⁴ | 2.2 × 10⁵ | 2.2 × 10⁵ | 3.8 × 10⁴ | 1.0 × 10⁵ | <1.0 × 10³ | <1.0 × 10³ |
| 413 | 2.8 × 10⁵ | N.T | 2.8 × 10⁵ | 2.8 × 10⁵ | 3.2 × 10⁴ | 7.9 × 10⁴ | <1.0 × 10³ | <1.0 × 10³ |
| 418 | 2.2 × 10⁵ | 4.3 × 10³ | 2.2 × 10⁵ | 2.2 × 10⁵ | <1.0 × 10³ | 6.3 × 10⁴ | <1.0 × 10³ | <1.0 × 10³ |
| 601 | 2.4 × 10⁵ | 1.0 × 10⁴ | 2.4 × 10⁵ | 2.4 × 10⁵ | 6.1 × 10⁴ | 1.4 × 10⁵ | 3.0 × 10⁴ | 3.1 × 10⁴ |
| 602 | 1.2 × 10⁵ | 3.2 × 10⁴ | 1.2 × 10⁵ | 1.2 × 10⁵ | 5.2 × 10⁴ | 1.4 × 10⁵ | 2.4 × 10⁴ | 2.9 × 10⁴ |

N.T: not analyzed

TABLE 3
(2) Binding constant for sugar chains having fucose other than α1→6 fucose (at 25° C.)

| Sugar | | | | | | | | |
|---|---|---|---|---|---|---|---|---|
| chain | PhoSL | SRL | NSL | LSL | AAL | AOL | LCL | PSL |
| No. | (M⁻¹) | (M⁻¹) | (M⁻¹) | (M⁻¹) | (M⁻¹) | (M⁻¹) | (M⁻¹) | (M⁻¹) |
| 419 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 420 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 718 | <1.0 × 10³ | <1.0 × 10³ | <1.0 × 10³ | <1.0 × 10³ | 6.5 × 10⁴ | 1.7 × 10^{s} | <1.0 × 10³ | <1.0 × 10³ |
| 719 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 720 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 721 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 722 | <1.0 × 10³ | <1.0 × 10³ | <1.0 × 10³ | <1.0 × 10³ | <1.0 × 10³ | 5.6 × 10⁴ | <1.0 × 10³ | <1.0 × 10³ |
| 723 | <1.0 × 10³ | <1.0 × 10³ | <1.0 × 10³ | <1.0 × 10³ | 3.5 × 10⁴ | <1.0 × 10³ | <1.0 × 10³ | <1.0 × 10³ |
| 726 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 727 | <1.0 × 10³ | <1.0 × 10³ | <1.0 × 10³ | <1.0 × 10³ | 1.3 × 10⁵ | 7.6 × 10⁴ | <1.0 × 10³ | <1.0 × 10³ |
| 728 | <1.0 × 10³ | <1.0 × 10³ | <1.0 × 10³ | <1.0 × 10³ | <1.0 × 10³ | 1.2 × 10⁴ | <1.0 × 10³ | <1.0 × 10³ |
| 729 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 730 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 731 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 739 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 909 | <1.0 × 10³ | <1.0 × 10³ | <1.0 × 10³ | <1.0 × 10³ | 3.1 × 10⁵ | 4.4 × 10⁴ | <1.0 × 10³ | <1.0 × 10³ |
| 910 | <1.0 × 10³ | <1.0 × 10³ | <1.0 × 10³ | <1.0 × 10³ | 9.0 × 10⁴ | 5.6 × 10⁴ | <1.0 × 10³ | <1.0 × 10³ |
| 931 | <1.0 × 10³ | <1.0 × 10³ | <1.0 × 10³ | <1.0 × 10³ | <1.0 × 10³ | 9.9 × 10⁴ | <1.0 × 10³ | <1.0 × 10³ |
| 932 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 933 | <1.0 × 10³ | <1.0 × 10³ | <1.0 × 10³ | <1.0 × 10³ | 2.1 × 10⁵ | 3.1 × 10⁴ | <1.0 × 10³ | <1.0 × 10³ |

TABLE 4
(3) Binding constant for sugar chains having no fucose (at 25° C.)

| Sugar | | | | | | | | |
|---|---|---|---|---|---|---|---|---|
| chain | PhoSL | SRL | NSL | LSL | AAL | AOL | LCL | PSL |
| No. | (M⁻¹) | (M⁻¹) | (M⁻¹) | (M⁻¹) | (M⁻¹) | (M⁻¹) | (M⁻¹) | (M⁻¹) |
| 001 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 002 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 003 | <1.0 × 10³ | <1.0 × 10³ | <1.0 × 10³ | <1.0 × 10³ | <1.0 × 10³ | <1.0 × 10³ | 7.2 × 10³ | 7.8 × 10³ |
| 004 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 005 | <1.0 × 10³ | <1.0 × 10³ | <1.0 × 10³ | <1.0 × 10³ | <1.0 × 10³ | 7.3 × 10³ | <1.0 × 10³ | 7.4 × 10³ |
| 006 | <1.0 × 10³ | <1.0 × 10³ | <1.0 × 10³ | <1.0 × 10³ | <1.0 × 10³ | <1.0 × 10³ | 1.4 × 10⁴ | 7.1 × 10³ |
| 007 | <1.0 × 10³ | <1.0 × 10³ | <1.0 × 10³ | <1.0 × 10³ | <1.0 × 10³ | <1.0 × 10³ | <1.0 × 10³ | 7.8 × 10³ |
| 008 | <1.0 × 10³ | <1.0 × 10³ | <1.0 × 10³ | <1.0 × 10³ | <1.0 × 10³ | 6.9 × 10⁴ | 1.8 × 10⁴ | 1.0 × 10⁴ |
| 009 | <1.0 × 10³ | <1.0 × 10³ | <1.0 × 10³ | <1.0 × 10³ | <1.0 × 10³ | <1.0 × 10³ | 1.8 × 10⁴ | 7.9 × 10³ |
| 010 | <1.0 × 10³ | <1.0 × 10³ | <1.0 × 10³ | <1.0 × 10³ | <1.0 × 10³ | <1.0 × 10³ | 1.3 × 10⁴ | <1.0 × 10³ |
| 011 | <1.0 × 10³ | <1.0 × 10³ | <1.0 × 10³ | <1.0 × 10³ | <1.0 × 10³ | <1.0 × 10³ | 1.4 × 10⁴ | <1.0 × 10³ |
| 012 | <1.0 × 10³ | <1.0 × 10³ | <1.0 × 10³ | <1.0 × 10³ | <1.0 × 10³ | <1.0 × 10³ | 2.5 × 10⁴ | 1.7 × 10⁴ |
| 013 | <1.0 × 10³ | <1.0 × 10³ | <1.0 × 10³ | <1.0 × 10³ | <1.0 × 10³ | <1.0 × 10³ | 1.7 × 10⁴ | <1.0 × 10³ |
| 014 | <1.0 × 10³ | <1.0 × 10³ | <1.0 × 10³ | <1.0 × 10³ | <1.0 × 10³ | <1.0 × 10³ | 1.7 × 10⁴ | 8.6 × 10³ |
| 101 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 103 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 104 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 105 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 107 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 108 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 301 | <1.0 × 10³ | <1.0 × 10³ | <1.0 × 10³ | <1.0 × 10³ | <1.0 × 10³ | 1.8 × 10⁴ | <1.0 × 10³ | <1.0 × 10³ |
| 304 | <1.0 × 10³ | <1.0 × 10³ | <1.0 × 10³ | <1.0 × 10³ | <1.0 × 10³ | 2.0 × 10⁴ | <1.0 × 10³ | <1.0 × 10³ |
| 305 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 307 | <1.0 × 10³ | <1.0 × 10³ | <1.0 × 10³ | <1.0 × 10³ | <1.0 × 10³ | <1.0 × 10³ | <1.0 × 10³ | 2.4 × 10³ |
| 308 | <1.0 × 10³ | <1.0 × 10³ | <1.0 × 10³ | <1.0 × 10³ | <1.0 × 10³ | 2.0 × 10⁴ | <1.0 × 10³ | <1.0 × 10³ |
| 313 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 314 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 323 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 501 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 502 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 503 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 504 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |

TABLE 5
(4) Binding constant for sugar chains having no fucose (at 25° C.)

| Sugar | | | | | | | | |
|---|---|---|---|---|---|---|---|---|
| chain | PhoSL | SRL | NSL | LSL | AAL | AOL | LCL | PSL |
| No. | (M⁻¹) | (M⁻¹) | (M⁻¹) | (M⁻¹) | (M⁻¹) | (M⁻¹) | (M⁻¹) | (M⁻¹) |
| 701 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 702 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 703 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 704 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 705 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 706 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 707 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 708 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 709 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 710 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 711 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 712 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 713 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 715 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 716 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 717 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 724 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 725 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 728 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 732 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 733 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 734 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 735 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 736 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 737 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 738 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 901 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 902 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 903 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 905 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 906 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |
| 907 | <1.0 × 10³ | <1.0 × 10³ | <1.0 × 10³ | <1.0 × 10³ | <1.0 × 10³ | <1.0 × 10³ | <1.0 × 10³ | <1.0 × 10³ |

The AAL and AOL bind to the fucose α1→6 sugar chains (sugar chains No. 015, 201 to 203, and 401 to 418), as well as to the glycolipid-type sugar chain excluding the fucose α1→6 (sugar chains No. 718, 722, 723, 727, 909, 910 and 933). The LCL and PSL bind to the fucose α1→6 sugar chain, as well as to sugar chain excluding the α1→6 fucose (sugar chains No. 003, and 005 to 014).

The fucose α1→6 specific lectin such as PhoSL firmly binds to the fucose α1→6 sugar chain and does not bind to the sugar chain excluding the α1→6 fucose at all. Moreover, its coupling constant (at 25° C.) is larger than that of the conventional fucose α1→6 affinitive lectin (coupling constant is 1.0×10⁴ M⁻¹ or more). Furthermore, the binding constant of the fucose α1→6 specific lectin is not decreased even if a sialic acid is added to the fucose α1→6 sugar chain (sugar chains No. 601 and 602). In addition, the fucose α1→6 specific lectin also strongly binds to the triple-strand (sugar chains No. 407 to 413) and the four-strand (sugar chains No. 418) of the fucose uα1→6 sugar chain.

The method according to the present invention specifically includes the following steps:

(A) reacting a fucosylated PSA contained in a specimen consisting of serum sampled from a subject with a fucose α1→6 specific lectin to obtain a fucosylated PSA-fucose α1→6 specific lectin complex; and

(B) detecting the complex by an appropriate means.

As described in the following Reference Example 1, when the fucose α1→6 specific lectin is made to actuate on the fucosylated PSA in serum sampled from a subject, there are problems such as high noise due to serum impurities. The method according to the present invention improves the sensitivity of the fucosylated PSA-fucose α1→6 specific lectin complex by adjusting a pH to within a specific alkaline range in at least one of the steps (A) and (B).

Specifically, the pH is adjusted to within a specific alkaline range, for at least one solution selected from: a solvent for the lectin reaction step of reacting the fucosylated PSA with the fucose α1→6 specific lectin to obtain the fucosylated PSA-fucose α1→6 specific lectin complex; a lavage fluid for a washing step of washing the complex; a solvent for a probe reaction step for reacting the complex with a secondary probe or subsequent probes; and a lavage fluid for washing the complex after the probe reaction. Preferably, the pH in the lectin reaction step is adjusted to within a specific alkaline range.

The lower limit of the pH in the alkaline region is higher than 8.5, preferably 8.6 or higher, more preferably 8.8 or higher, further preferably 9.0 or higher. The upper limit of the pH in the alkaline region is lower than 11.0, preferably 10.5 or lower. If the pH is 8.5 or lower or 11.0 or higher, a signal/noise ratio of the complex cannot be improved in some cases.

The pH is adjusted by adding an alkaline reagent, preferably an alkaline solution, more preferably an alkaline buffer. Examples of the alkaline buffer include a glycine-sodium hydroxide (NaOH) buffer; a carbonate-bicarbonate buffer; a Good's buffer such as TAPS, Tricine, Bicine, CHES, CAPSO, and CAPS; a sodium borate buffer; an ammonium chloride buffer; a wide range buffer such as Britton-Robinson buffer; and the like. At least one selected from the glycine-NaOH buffer, the carbonate-bicarbonate buffer, and the TAPS buffer is preferable, and at least one selected from the glycine-NaOH buffer and the TAPS buffer is more preferable. Preparation of these buffers is based on conventionally known methods.

It is preferable to add albumin, e.g., bovine serum albumin (BSA) to the reaction between the fucosylated PSA and the fucose α1→6 specific lectin in the step (A). An albumin concentration in the reaction solution may be normally 0.01 to 10% and is preferably 0.1 to 3%, particularly preferably 0.5 to 1%.

Preferably, a labeling means is previously incorporated in the lectin for detecting the complex in the step (B). The labeling means is not particularly limited but a known labeling method can be applied, and examples of the method include labeling with a radioisotope, binding of a labeling compound, and the like. Examples of the radioisotope include ¹⁴C, ³H and ³²P. Also, an anti-lectin antibody capable of binding to the lectin may be used for detection.

Example of the labeling compound include an enzyme label (horseradish peroxidase, alkaline phosphatase, etc.), a biotin label, a digoxigenin label, and a fluorescent label (fluorescein isothiocyanate, CyDye (registered trademark), ethyl 4-aminobenzoate (ABEE), aminopyridine, allophycocyanin, phycoerythrin, Alexa Fluor (registered trademark), etc.). These labeling compounds can be bound to the lectin by a ordinary method. In particular, the biotin label is preferred for its high sensitivity.

In the step (B), the means for detecting the complex is not particularly limited. As the detection means, ELISA (direct adsorption method, sandwich method and competition method), lectin affinity chromatography, lectin staining, lectin chip, flow cytometry (FACS) method, coagulation method, surface plasmon resonance method (e.g., Biacore (registered trademark) system), electrophoresis, beads, and the like can be used. Several representative detection methods are outlined below.

In the direct adsorption ELISA method, a specimen (serum) is added to a plate and immobilized. Then, the biotin-labeled lectin is added to allow the PSAto react with the lectin. As a secondary labeling compound, an HRP (horseradish peroxidase)-labeled streptavidin solution is added to allow the biotin to react with the streptavidin. Subsequently, a chromogenic substrate for HRP is added to develop color, and the coloring intensity is measured with an absorptiometer. The sugar chain can also be quantified by previously graphing a calibration curve with a standard sample containing a known concentration of the sugar chain.

In the sandwich ELISA method, at least one selected from lectins and antibodies (e.g., anti-PSA antibody) or fragments thereof having affinity for the fucosylated PSA are added to a plate and immobilized. The antibody may be either a monoclonal antibody or a polyclonal antibody. Then, a specimen (serum) is exposed to the plate. Then, the biotin-labeled fucose α1→6 specific lectin is added to allow the fucosylated PSA in the serum to react with the fucose α1→6 specific lectin. This reaction produces the complex of the fucosylated PSA and the fucose α1→6 specific lectin. As a secondary labeling compound, an HRP-labeled streptavidin solution is added to allow the biotin to react with the streptavidin. Subsequently, a chromogenic substrate for HRP is added to develop color, and the coloring intensity is measured with an absorptiometer. The α1→6 fucose sugar chain can also be quantified by previously graphing a calibration curve with a standard sample of a known concentration.

The lectin affinity chromatography is an affinity chromatography utilizing the property that a lectin immobilized on a carrier specifically binds to a sugar chain. High throughput can be expected by combining with HPLC.

As a carrier for immobilizing the lectin, gel materials such as agarose, dextran, cellulose, starch and polyacrylamide are commonly used. For these materials, commercial products can be used without special limitation, and exemplified by Sepharose 4B and Sepharose 6B (both from GE Healthcare Biosciences Corp.). Examples of a column used for the lectin chromatography also include a column prepared by immobilizing the lectin on a microplate or a nanowell.

A concentration of a lectin to be immobilized is generally 0.001 to 100 mg/mL, preferably 0.01 to 20 mg/mL. When the carrier is an agarose gel, it is activated with CNBr or the like and then coupled with the lectin. The lectin may be immobilized on a gel into which the activated spacer has been introduced. Furthermore, the lectin may be immobilized on a gel into which a formyl group has been introduced and then reduced with NaCNBH₃. Alternatively, a commercial activated gel such as NHS-Sepharose (from GE Healthcare Biosciences Corp.) may be used.

The specimen (serum) is put in a column, to which subsequently a buffer solution is shed for the purpose of washing. Alternatively, the specimen in the buffer solution is put in the column. The buffer solution can be exemplified by a phosphate buffer solution, a tris buffer solution, a glycine buffer solution and the like, and it has a molar concentration of generally 5 to 500 mM, preferably 10 to 500 mM, and a pH of generally 4.0 to 10.0, preferably 6.0 to 9.0. In addition, it is a buffer solution in which a content of NaCl is generally 0 to 0.5 M, preferably 0.1 to 0.2 M, and a content of CaCl₂, MgCl₂ or MnCl₂ is generally 0 to 10 mM, preferably 0 to 5 mM.

After washing the affinity column, the sugar chain is eluted in a neutral non-modified buffer solution capable of effectively eluting the sugar chain using a desorbent such as sodium chloride and hapten sugar. This buffer solution may be the same as described above. The concentration of the desorbent is preferably 1 to 500 mM, particularly preferably 10 to 200 mM.

In step (B), a signal (reaction value) from the complex of the fucosylated PSA in the serum and the fucose α1→6 specific lectin is compared with a signal (reference value) obtained in a person having a Gleason score of 6 or less, preferably 6, to evaluate with higher accuracy the presence or absence of high-risk prostate cancer development and, if cancer has been developed, its malignancy. That means, when a signal (reaction value) of a specimen is higher than a signal (reference value) obtained from a person with a Gleason score of 6 or lower, it is suggested that a subject of the specimen has a high-risk prostate cancer.

The level of the signal (reaction value) from the complex of the fucosylated PSA in the serum and the fucose α1→6 specific lectin depends on the reaction condition of the lectin, the PSA concentration of the blood fucosylated PSA, and the type of the lectin. A calibration curve expressing the relationship between the fucosylated PSA concentration and the signal value is graphed using a fucosylated PSA reference standard (known concentration) so as to quantify the signal. For each lectin, a reaction value corresponding to the fucosylated PSA concentration of 10 ng/mL is taken as 10 U/mL.

The present invention also provides a diagnostic agent for detecting prostate cancer containing: a fucose α1→6 specific lectin; and an alkaline reagent for adjusting a pH to within a specific alkaline region in at least one step selected from a group of steps including a step for reacting a fucosylated PSA contained in a specimen consisting of serum sampled from a subject with a fucose α1→6 specific lectin, and subsequent processing steps. Explanation of the fucose α1→6 specific lectin and the alkaline reagent is as described above.

The diagnostic agent may appropriately include agents generally used for detection, such as various labeling compounds, a buffer, a plate, beads and a reaction-stopping liquid. The diagnostic agent preferably includes a reagent for extracting a fucosylated PSA contained in a specimen consisting of serum (e.g., an anti-PSA antibody, or a fragment or analogue thereof).

### EXAMPLE

Hereinafter, the present invention will be described in more detail with reference to Examples of the present invention. However, the present invention is not limited to the following Examples.

### 1. Preparation of Sample

Reagents used for the detection method of the present invention were prepared by the following procedure.

### (1) Anti-PSA Antibody for Solid-Phasing

An anti-PSA antibody was purchased from HyTest, Ltd. and used as an anti-PSA antibody for solid phasing after removing sugar chains thereof in accordance with the method described in Non-Patent Document 5.

### (2) Fucosylated PSA Reference Standard

A fucosylated PSA was purified from a PSA (reference standard, from BBI Solutions) using an LCA (Lens culinaris lectin) column (from J-OIL MILLS, Inc.) to obtain a fucosylated PSA reference standard.

### (3) Biotin-Labeled Lectin

As the fucose α1→6 specific lectin used in the method according to the present invention, Pholiota squarrosa lectin (PhoSL), Stropharia rugosoannulata lectin (SRL), Naematoloma sublateritium lectin (NSL), Amanita muscaria lectin (AML), and Pholiota squarrosa lectin peptide (hereinafter referred to as PhoSL peptide, SEQ ID No. 6) were prepared. This PhoSL peptide was synthesized on the basis of the description of Example 6 in Patent Document 3 (with the proviso that PTL in Patent Document 3 is replaced with PhoSL). These lectins were weighed out, to which a 0.1 M sodium bicarbonate solution was added for dissolution (concentration: 5 mg/mL). A biotinylating reagent dissolved in dimethylsulfoxide was added to the lectin solution and reacted. The reaction solution was subjected to solvent substitution with water using ultrafiltration (molecular weight cut-off: 3 K). This solution was lyophilized to obtain a biotin-labeled lectin. In addition, a biotin-labeled Aleuria aurantia lectin (AAL, from J-OIL MILLS, Inc.) was prepared as an α1→6 fucose affinitive lectin.

### (4) Reagents and the Like for Use (4-1) Phosphate Buffered Saline (PBS)

5.75 g of disodium hydrogenphosphate, 1.0 g of potassium dihydrogenphosphate, 1.0 g of potassium chloride, and 40.0 g of sodium chloride were dissolved in 5 L of water to obtain PBS (pH 7.4).

### (4-2) 100 mM Glycine-Sodium Hydroxide Buffer (Glycine-NaOH, pH 10)

3.76 g of glycine was dissolved in about 400 mL of water. 5N sodium hydroxide was added thereto to adjust the pH to 10. Then, the volume was adjusted to 500 mL by adding water to prepare a buffer of pH 10.

### (4-3) 1% Bovine Serum Albumin (BSA)/PBS

1 g of bovine serum albumin (BSA, from Sigma-Aldrich Co. LLC) was dissolved in 100 mL of PBS to obtain a PBS solution with a BSA concentration of 1% (hereinafter referred to as 1% BSA/PBS).

### (4-4) 0.1% Bovine Serum Albumin (BSA)/PBS

0.1 g of bovine serum albumin was dissolved in 100 mL of PBS to obtain a PBS solution with a BSA concentration of 0.1% (hereinafter referred to as 0.1% BSA/PBS).

### (4-5) 0.1% BSA/10-Fold Diluted PBS+Glycine-NaOH (pH 9.6)

To 5 mL of a solution obtained by 5-fold dilution of 1% BSA/PBS, 5 mL of the glycine-NaOH was added to obtain 0.1% BSA/10-fold diluted PBS+glycine-NaOH (pH 9.6).

### (5-1) Subject Sample A

Human serum specimens sampled from 9 patients diagnosed with prostate cancer and 7 healthy persons were purchased from KAC Co., Ltd. and used as subject samples A. Blood PSA levels of the respective specimens, as well as blood PSA levels and prostate cancer risk classification of the respective prostate cancer patients are shown in Table 6.

TABLE 6

| | | Blood PSA | | | | | |
|---|---|---|---|---|---|---|---|
| | | value | Diagnosis of | Histological | | TNM | |
| Name | Age | (ng/mL) | patient | diagnosis | GS | classification | Staging |
| Healthy 1 | U/A | U/A | Healthy | — | — | — | — |
| Healthy 2 | U/A | U/A | Healthy | — | — | — | — |
| Healthy 3 | U/A | U/A | Healthy | — | — | — | — |
| Healthy 4 | U/A | U/A | Healthy | — | — | — | — |
| Healthy 5 | U/A | U/A | Healthy | — | — | — | — |
| Healthy 6 | U/A | U/A | Healthy | — | — | — | — |
| Healthy 7 | U/A | U/A | Healthy | — | — | — | — |
| Prostate | 76 | 4.49 | Prostate | Adenocarcinoma | 4 (2 + 2) | T2NxM0 | II |
| cancer 1 | | | cancer | | | | |
| Prostate | 79 | 19.00 | Prostate | Adenocarcinoma | 5 (2 + 3) | T2NxM0 | II |
| cancer 2 | | | cancer | | | | |
| Prostate | 80 | 2.82 | Prostate | Adenocarcinoma | 6 (3 + 3) | T2NxM0 | II |
| cancer 3 | | | cancer | | | | |
| Prostate | 66 | 24.11 | Prostate | Adenocarcinoma | 6 (3 + 3) | T2cN0M0 | IIB |
| cancer 4 | | | cancer | | | | |
| Prostate | 67 | 16.95 | Prostate | Adenocarcinoma | 7 (3 + 4) | T2cN0M0 | IIB |
| cancer 5 | | | cancer | | | | |
| Prostate | 80 | 156 | Prostate | Adenocarcinoma | 7 (3 + 4) | T2N0M1 | IV |
| cancer 6 | | | cancer | | | | |
| Prostate | 51 | 3.54 | Prostate | Adenocarcinoma | 8 (3 + 5) | T2cN0M0 | IIB |
| cancer 7 | | | cancer | | | | |
| Prostate | 64 | 7.82 | Prostate | Adenocarcinoma | 8 (4 + 4) | T2cN0M0 | IIB |
| cancer 8 | | | cancer | | | | |
| Prostate | 54 | 477 | Prostate | Adenocarcinoma | 8 (4 + 4) | T2N0M1 | IV |
| cancer 9 | | | cancer | | | | |

U/A: not available

### (5-2) Subject Sample B

Informed consent was gotten from patients with prostate cancer at Osaka University Hospital, and then their collected serum was used as subject samples B. Information on the respective specimen is shown in Table 7 with classification on the basis of their Gleason scores. The “Negative” in Table 7 refers to a group in which the blood PSA level was high but prostate cancer was not detected in the prostate biopsy.

TABLE 7

| Group | n | Age | GS |
|---|---|---|---|
| Negative | 9 | 59-76 | — |
| G6 | 9 | 60-78 | 6 |
| G7 | 11 | 55-74 | 7 |
| G8-9 | 6 | 59-76 | 8-9 |

### [Example 1] Fucosylated PSA Detecting Test Using Pholiota squarrosa Lectin (PhoSL) (I)

For the purpose of detecting the serum fucosylated PSA of prostate cancer patients with high sensitivity, a test was executed in which the pH was changed during the reaction between the serum fucosylated PSA and PhoSL.

### 1. Sandwich ELISA

### (1) Antibody Immobilization

The anti-PSA antibody from which sugar chains had been removed was diluted to 5 μg/mL with PBS. 25 μL of this diluted solution was added to each well of an ELISA plate and allowed to stand at 37° C. for 12 hours, and then the additive solution was discarded.

### (2) Washing

150 μL of PBS containing 0.05% Tween 20 (product name: polyoxyethylene sorbitan monolaurate, from Nacalai Tesque, Inc.) was added to each well, and then the additive solution was discarded. This manipulation was repeated three times in total.

### (3) Blocking

25 μL of 1% BSA/PBS was added to each well and allowed to stand at 37° C. for 1 hour, and then the additive solution was discarded.

### (4) Washing

150 μL of PBS containing 0.05% Tween 20 was added to each well, and then the additive solution was discarded. This manipulation was repeated three times in total.

### (5) Antigen-Antibody Reaction

To graph a calibration curve, 25 μL of the fucosylated PSA reference standard diluted with 1% BSA/PBS to a concentration of 0 to 200 ng/mL was added to each well and allowed to stand at room temperature for 1 hour, and then the additive solution was discarded. In addition, to detect the fucosylated PSA in the serum of the subject by the lectin, 25 μL of serum of “Healthy 1” and “Prostate cancer 4” 2-fold diluted with PBS was added to each well and allowed to stand at room temperature for 1 hour, and then the additive solution was discarded.

### (6) Washing

150 μL of PBS containing 0.05% Tween 20 was added to each well, and then the additive solution was discarded. This manipulation was repeated three times in total.

### (7) Reaction of Labeled Lectin

In Reference Example 1, 25 μL of a biotin-labeled PhoSL diluted to 0.1 μg/mL with 0.1% BSA/PBS (pH 7.4) was added to each well and allowed to stand at 4° C. for 30 minutes, and then the additive solution was discarded. In Example 1, 25 μL of a biotin-labeled PhoSL diluted to 0.1 μg/mL with 0.1% BSA/10-fold diluted PBS+glycine-NaOH (pH 9.6) was added to each well and allowed to stand at 4° C. for 30 minutes, and then the additive solution was discarded.

### (8) Washing

150 μL of PBS containing 0.05% Tween 20 was added to each well, and then the additive solution was discarded. This manipulation was repeated three times in total.

### (9) HRP-Labeled Streptavidin Reaction

25 μL of a horseradish peroxidase (HRP)-labeled streptavidin solution (from Vector Laboratories, Inc., concentration: 0.4 μg/mL, in 1% BSA/PBS) was added to each well and allowed to stand at room temperature for 30 minutes, and then the additive solution was discarded.

### (10) Washing

150 μL of PBS containing 0.05% Tween 20 was added to each well, and then the additive solution was discarded. This manipulation was repeated three times in total.

### (11) Coloring Reaction

25 μL of chromogenic substrate for HRP (product name: TMB, from Kirkegaard & Perry Laboratories, Inc.) was added to each well and allowed to stand at room temperature for 10 minutes.

### (12) Termination of Reaction

25 μL of 1M phosphoric acid was added to terminate the reaction.

### (13) Absorbance Measurement

Absorbance (Ab) at 450 nm and 630 nm was measured using a plate reader, and a measurement (Ab₄₅₀₋₆₃₀) was obtained.

### (14) Graphing of Calibration Curve

A calibration curve was graphed by plotting signals (reaction values, Ab₄₅₀₋₆₃₀) of the fucosylated PSA reference standard with the biotin-labeled PhoSL. A Signal (Ab₄₅₀₋₆₃₀) of the biotin-labeled PhoSL corresponding to the fucosylated PSA concentration of 10 ng/mL was defined as 10 U/mL.

### (15) Calculation of Reaction Value

The signals of biotin-labeled PhoSL (Ab₄₅₀₋₆₃₀) for the serum fucosylated PSA in the subject samples A were assigned to the calibration curve to calculate reaction values (unit: U/mL).

Table 8 shows results of measuring the PhoSL reaction values in tests (Reference Example 1 and Example 1) in which the pH in the lectin reaction was changed.

TABLE 8

| | Reference Example 1 | Example 1 |
|---|---|---|
| (7) pH in lectin reaction | 7.4 | 9.6 |
| PhoSL reaction value A of | 108.4 U/mL | 178.8 U/mL |
| Prostate cancer 4 (GS6) | | |
| PhoSL reaction value B of | 35.8 U/mL | 51.9 U/mL |
| Healthy 1 | | |
| Δ (=A − B) | 72.6 U/mL | 126.9 U/mL |

Table 8 shows that in Example 1 in which the pH was adjusted to within a specific alkaline range according to the present invention in the reaction step between the serum fucosylated PSA and the PhoSL, the PhoSL reaction value A of prostate cancer increases, meanwhile the PhoSL reaction value B of “healthy 1” decreases, compared to Reference Example 1. As a result of increase in the difference A between the reaction values A and B, it turned out that a prostate cancer patient (GS6) could be detected with high sensitivity in Example 1 according to the present invention.

### [Example 2] Fucosylated PSA Detecting Test Using PhoSL (II)

The PhoSL reaction values for the subject samples A were measured by the same operation as in Example 1. The results are shown in Table 9. From the PhoSL reaction values, an average value and a median value were determined. Furthermore, a standard cutoff value (89.7 U/mL) was used to determine a detection rate (positive rate) of the prostate cancer patients, and a false detection rate (false positive rate) of the healthy persons. The results are shown in Table 10.

Comparative Example 1 in Table 9 indicates results of measuring the blood PSA values. A cut-off value of the serum PSA value was 4 ng/mL. As in Example 2, an average value, a median value, a positive rate and a false positive rate were determined. The results are shown in Table 10.

Comparative Example 2 in Table 9 indicates results of detecting the serum fucosylated PSA using AAL (AAL reaction values) in the subject samples A. Specifically, in Reference Example 1, AAL reaction values were measured in the same manner as in Reference Example 1 except that the biotin-labeled PhoSL was replaced with a biotin-labeled AAL. A standard cut-off value for the AAL reaction values was 894.8 U/mL. As in Example 2, an average value, a median value, a positive rate and a false positive rate were determined from the AAL reaction values. The results are shown in Table 10.

TABLE 9

| | | | | Comparative | |
|---|---|---|---|---|---|
| | | | Comparative | Example 2 | Example 2 |
| | | | Example 1 | AAL | PhoSL |
| | | | Blood PSA | reaction | reaction |
| | | | value | value | value |
| | | GS | (ng/mL) | (U/mL) | (U/mL) |
| Healthy | Healthy 1 | — | N/A | 1099.6 | 51.9 |
| person | Healthy 2 | — | N/A | 854.6 | 55.9 |
| group | Healthy 3 | — | N/A | 1062.7 | 63.9 |
| | Healthy 4 | — | N/A | 115.0 | 74.9 |
| | Healthy 5 | — | N/A | 32.3 | 55.8 |
| | Healthy 6 | — | N/A | 1193.1 | 39.7 |
| | Healthy 7 | — | N/A | 1028.8 | 59.0 |
| Prostate | Prostate | 4 (2 + 2) | 4.49 | 831.2 | 115.8 |
| cancer | cancer 1 | | | | |
| patient | Prostate | 5 (2 + 3) | 19.00 | 1032.3 | 116.7 |
| group | cancer 2 | | | | |
| | Prostate | 6 (3 + 3) | 2.82 | 1152.7 | 104.5 |
| | cancer 3 | | | | |
| | Prostate | 6 (3 + 3) | 24.11 | 926.5 | 178.8 |
| | cancer 4 | | | | |
| | Prostate | 7 (3 + 4) | 16.95 | 1025.0 | 115.8 |
| | cancer 5 | | | | |
| | Prostate | 7 (3 + 4) | 156.00 | 961.5 | 167.5 |
| | cancer 6 | | | | |
| | Prostate | 8 (3 + 5) | 3.54 | 1211.9 | 125.6 |
| | cancer 7 | | | | |
| | Prostate | 8 (4 + 4) | 7.82 | 1010.0 | 129.8 |
| | cancer 8 | | | | |
| | Prostate | 8 (4 + 4) | 477.00 | 988.1 | 251.4 |
| | cancer 9 | | | | |

TABLE 10

| | | | Comparative | |
|---|---|---|---|---|
| | | Comparative | Example 2 | Example 2 |
| | | Example 1 | AAL | PhoSL |
| | | Blood PSA | reaction | reaction |
| | | value | value | value |
| Healthy | Average value | — | 769.4 U/mL | 57.3 U/mL |
| person | Median value | — | 1028.8 U/mL | 55.9 U/mL |
| group | False positive | — | 57% | 0% |
| | rate | | | |
| Prostate | Average value | 69.5 ng/mL | 1015.5 U/mL | 145.1 U/mL |
| cancer | Median value | 12.4 ng/mL | 1010.0 U/mL | 125.6 U/mL |
| patient | Positive rate | 77% | 89% | 100% |
| group | | | | |

As shown in Table 10, a detection rate (positive rate) of the prostate cancer patient group based on the blood PSA values was 77%. A detection rate (positive rate) of the prostate cancer patient group based on the AAL reaction values was 89%. However, also a false positive rate obtained by judging a healthy group to have prostate cancer was as high as 57%. In Example 2 according to the present invention, a positive rate of the prostate cancer patient group was 100%, and a false positive rate of the healthy group was 0%. From the above description, it was revealed that when the fucose α1→6 specific lectin was used under a specific condition according to the present invention, prostate cancer could be detected with high sensitivity and high specificity.

Next, the measurement results of the prostate cancer patient group in Table 9 were classified into Negative (G5 or lower), GS6, GS7, and GS8 or higher. An average value and a median value of each group are shown in Table 11.

TABLE 11

| | | | Comparative | |
|---|---|---|---|---|
| | | Comparative | Example 2 | Example 2 |
| | | Example 1 | AAL | PhoSL |
| | | Blood PSA | reaction | reaction |
| | | value | value | value |
| Subgroup | n | (ng/mL) | (U/mL) | (U/mL) |
| Negative | 2 | 11.7 | 931.8 | 116.3 |
| (GS5 or less) | | 11.7 | 931.8 | 116.3 |
| GS6 | 2 | 13.5 | 1039.6 | 141.7 |
| | | 13.5 | 1039.6 | 141.7 |
| GS7 | 2 | 86.5 | 993.3 | 141.7 |
| | | 86.5 | 993.3 | 141.7 |
| GS8 or more | 3 | 162.8 | 1070.0 | 129.8 |
| | | 7.8 | 1010.0 | 168.9 |

Upper line: Mean value
Lower line: Median value

As shown in Table 11, no correlation is observed between the median blood PSA value or the median AAL reaction value and the GS. In Example 2, there is a tendency of increase in the GS (malignant progression) and in the median PhoSL reaction value.

### [Example 3] Fucosylated PSA Detecting Test Using PhoSL (III)

For the purpose of confirming the presence of a correlation between the median PhoSL reaction value and the GS in Example 2, the PhoSL reaction values were measured in subject samples B having an n number more than that in the subject samples A in the same operation as in Example 2. The results are shown in Table 12. For comparison, the results of measuring the blood PSA values are also shown in Table 12.

TABLE 12

| | | Comparative Example 3 | Example 3 |
|---|---|---|---|
| | | Blood PSA value | PhoSL reaction value |
| Subgroup | n | (ng/mL) | (U/mL) |
| Negative | 9 | 8.4 | 203.8 |
| | | 7.8 | 178.0 |
| | | 4.0 | 84.3 |
| GS6 | 9 | 17.4 | 181.7 |
| | | 6.8 | 188.9 |
| | | 29.4 | 31.88 |
| GS7 | 11 | 23.1 | 119.5 |
| | | 9.6 | 210.6 |
| | | 39.0 | 27.2 |
| GS8-9 | 6 | 75.3 | 232.9 |
| | | 12.9 | 239.8 |
| | | 156.4 | 52.5 |

Upper line: Mean value
Middle line: Median value
Lower line: Standard deviation

As shown in Table 12, there is no correlation between the blood PSA values and the GS. On the other hand, in Example 3 according to the present invention, the median PhoSL reaction value tends to increase as the GS increases. From the results in Tables 11 and 12, the GS can be predicted from the reaction value by using the fucose α1→6 specific lectin such as PhoSL under a specific condition. Consequently, the method according to the present invention is expected to predict the GS without biopsy and to predict a high-risk prostate cancer at GS7 or higher.

### [Example 4] Fucosylated PSA Detecting Test Using Stropharia rugosoannulata Lectin (SRL)

Two types of SRL reaction values were measured by the same operation as in Example 1 and Reference Example 1 except that the PhoSL in Example 1 and Reference Example 1 was changed to the SRL. The results are shown in Table 13.

TABLE 13

| | Reference Example 2 | Example 4 |
|---|---|---|
| (7) pH in lectin reaction | 7.4 | 9.6 |
| SRL reaction value A | 146.3 U/mL | 234.5 U/mL |
| of Prostate cancer 4 | | |
| SRL reaction value B | 20.0 U/mL | 16.8 U/mL |
| of Healthy 1 | | |
| Δ (=A − B) | 126.3 U/mL | 217.7 U/mL |

Table 13 shows that in Example 4 in which the serum fucosylated PSA was reacted with the SRL in a specific alkaline region, the reaction values of the prostate cancer patients increase and meanwhile the reaction values of the healthy persons decrease compared to Reference Example 2. Thus, even if the SRL is used under a specific condition, the serum fucosylated PSA can be detected with high sensitivity. Therefore, the SRL is effective for detecting prostate cancer.

### [Example 5] Fucosylated PSA Detecting Test Using Naematoloma sublateritium (NSL)

Two types of NSL reaction values were measured by the same operation as in Example 1 and Reference Example 1 except that the PhoSL in Example 1 and Reference Example 1 was changed to the NSL. The results are shown in Table 14.

TABLE 14

| | Reference Example 3 | Example 5 |
|---|---|---|
| (7) pH in lectin reaction | 7.4 | 9.6 |
| NSL reaction value A of | 399.7 U/mL | 282.6 U/mL |
| Prostate cancer 4 | | |
| NSL reaction value B of | 177.7 U/mL | 42.3 U/mL |
| Healthy 1 | | |
| Δ (=A − B) | 222 U/mL | 240.3 U/mL |

Table 14 shows that in Example 5 in which the serum fucosylated PSA was reacted with the NSL in a specific alkaline region, a difference A between the reaction values of the prostate cancer patients and the reaction values of the healthy persons increases compared to Reference Example 3. Thus, even if the NSL is used under a specific condition, the serum fucosylated PSA can be detected with high sensitivity. Therefore, the NSL is effective for detecting prostate cancer.

### [Example 6] Fucosylated PSA Detecting Test Using Amanita muscaria Lectin (AML)

Two types of AML reaction values were measured by the same operation as in Example 1 and Reference Example 1 except that the PhoSL in Example 1 and Reference Example 1 was changed to the AML. The results are shown in Table 15.

TABLE 15

| | Reference Example 4 | Example 6 |
|---|---|---|
| (7) pH in lectin reaction | 7.4 | 9.6 |
| AML reaction value A of | 141.3 U/mL | 150.9 U/mL |
| Prostate cancer 4 | | |
| AML reaction value B of | 21.2 U/mL | Below detection limit |
| Healthy 1 | | |
| Δ (=A − B) | 120.1 U/mL | 150.9 U/mL |

Table 15 shows that in Example 6 in which the serum fucosylated PSA was reacted with the AML in a specific alkaline region, the reaction values of the prostate cancer patients increase and meanwhile the reaction values of the healthy persons decrease compared to Reference Example 4. Even if the AML is used under a specific condition, the serum fucosylated PSA can be detected with high sensitivity. Therefore, the AML is effective for detecting prostate cancer.

### [Example 7] Fucosylated PSA Detecting Test Using PhoSL Peptide

Two types of PhoSL peptide reaction values were measured by the same operation as in Example 1 and Reference Example 1 except that the PhoSL in Example 1 and Reference Example 1 was changed to the PhoSL peptide. The results are shown in Table 16.

TABLE 16

| | Reference Example 5 | Example 7 |
|---|---|---|
| (7) pH in lectin reaction | 7.4 | 9.6 |
| PhoSL peptide reaction | 101.5 U/mL | 234.4 U/mL |
| value A of Prostate | | |
| cancer 4 | | |
| PhoSL peptide reaction | 1.0 U/mL | Below detection limit |
| value B of Healthy 1 | | |
| Δ (=A − B) | 100.5 U/mL | 234.4 U/mL |

Table 16 shows that in Example 7 in which the serum fucosylated PSA was reacted with the PhoSL peptide in a specific alkaline region, the reaction values of the prostate cancer patients increase and meanwhile the reaction values of the healthy persons decrease compared to Reference Example 5. Even if the PhoSL peptide is used under a specific condition, the serum fucosylated PSA can be detected with high sensitivity. Therefore, the PhoSL peptide is effective for detecting prostate cancer.

### [Sequence Listing]

### NP2360 SEQUENCEYNP2360PCT_ST25.txt

1. A method for detecting prostate cancer, comprising reacting a fucosylated PSA contained in a specimen consisting of serum sampled from a subject with a fucose α1→6 specific lectin to detect the reacted lectin, wherein
a pH is adjusted within an alkaline region lower than 11.0 in at least one step of the group consisting of a step for reacting the fucosylated PSA with the lectin and subsequent processing steps.

2. The method for detecting prostate cancer according to claim 1, wherein the fucose α1→6 specific lectin is extracted from a basidiomycete belonging to Strophariaceae, Tricholomataceae, Amanitaceae or Polyporaceae.

3. The method for detecting prostate cancer according to claim 1, wherein the fucose α1→6 specific lectin is at least one of Pholiota squarrosa lectin, Pholiota terrestris lectin, Stropharia rugosoannulata lectin, Naematoloma sublateritium lectin, Lepista sordida lectin, and Amanita muscaria lectin.

4. The method for detecting prostate cancer according to claim 1, wherein the fucose α1→6 specific lectin is a protein or peptide containing:
(a) a protein or peptide consisting of an amino acid sequence represented by any of SEQ ID Nos. 1 to 5; or
(b) a protein or peptide consisting of an amino acid sequence represented by any of SEQ ID Nos. 1 to 5 in which one or more amino acids are deleted, inserted or substituted, and being functionally equivalent to a protein having the amino acid sequence represented by any of SEQ ID Nos. 1 to 5.

5. The method for detecting prostate cancer according to claim 1, wherein the fucose α1→6 specific lectin is labeled.

6. The method for detecting prostate cancer according to claim 1, wherein the fucosylated PSA is detected using the fucose α1→6 specific lectin and an antibody.

7. The method for detecting prostate cancer according to claim 1, wherein a reaction between the fucosylated PSA and the fucose α1→6 specific lectin is performed in presence of albumin.

8. The method for detecting prostate cancer according to claim 1, wherein when a subject shows a signal (reaction value) resulting from the reaction between the fucosylated PSA and the fucose α1→6 specific lectin higher than a signal (reference value) obtained from a person with a Gleason score of 6 or lower, it is suggested that the subject has a high-risk prostate cancer.

9. A diagnostic agent kit for detecting prostate cancer, containing:
a fucose α1→6 specific lectin; and
an alkaline reagent for adjusting a pH within an alkaline region lower than 11.0 in at least one step selected from a group of steps including a step for reacting a fucosylated PSA contained in a specimen consisting of serum sampled from a subject with the fucose α1→6 specific lectin and subsequent processing steps.

10. The diagnostic agent kit for detecting prostate cancer according to claim 9, further containing an anti-PSA antibody.

11. The method for detecting prostate cancer according to claim 1, wherein an alkaline region is not lower than pH 7.4.

12. The diagnostic agent for detecting prostate cancer according to claim 10, wherein an alkaline region is not lower than pH 7.4.
